(19) United States
(12) Patent Application Publication
(10) Pub. No.: US 2022/0160451 A1
(43) Pub. Date: May 26, 2022

(54) CRASH CART AUTOMATION SYSTEMS AND METHODS

(71) Applicant: S&S X-Ray Products, Inc., Houston, TX (US)

(72) Inventors: Nishat Patel, Houston, TX (US); Brian Shoenfeld, Cypress, TX (US)

(73) Assignee: S&S X-Ray Products, Inc., Houston, TX (US)

(21) Appl. No.: 16/953,856

(22) Filed: Nov. 20, 2020

(51) Int. Cl. A61B 50/13; A61B 5/00; A61B 5/046; A61B 5/0464

(52) U.S. Cl. CPC A61B 50/13; A61B 5/742; A61B 5/7475; A61B 2050/185; A61B 5/0464; A61B 2560/0456; A61B 2560/0437; A61B 5/046

(57) ABSTRACT

In an embodiment, a method includes receiving, by a user device, a code start trigger, where the user device is dockable on a crash cart and in communication with a controller disposed in relation to the crash cart. The method also includes, responsive to the code start trigger, initiating a code workflow, creating an event log for the code workflow, and determining a patient rhythm. The method also includes monitoring for real-time code events. The monitored real-time code events include patient intervention and a new patient rhythm. The method also includes, responsive to a determination that a real-time code event has occurred, executing programmed action that includes updating the event log.

### BACKGROUND

### Technical Field

The present disclosure relates generally to technology for emergency medicine more particularly, but not by way of limitation, to systems and methods for automating crash carts.

### History of Related Art

Medical facilities typically have internal codes for situations when someone has suffered a cardiac arrest or a similar potentially fatal condition. When such codes are given, time is of the essence. Hospital staff usually clear the corridors and direct visitors to stand aside as a crash cart is rushed to the scene. A team of physicians and nurses immediately tend to the patient using supplies in the crash cart.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In a general aspect, in an embodiment, a method includes receiving, by a user device, a code start trigger, where the user device is (lockable on a crash cart and in communication with a controller disposed in relation to the crash cart. The method also includes, responsive to the code start trigger, initiating a code workflow, creating an event log for the code workflow, and determining a patient rhythm. The method also includes monitoring for real-time code events. The monitored real-time code events include patient intervention and a new patient rhythm. The method also includes, responsive to a determination that a real-time code event has occurred, executing programmed action that includes updating the event log. Other embodiments of this aspect include corresponding computer systems, apparatus, and computer programs recorded on one or more computer storage devices, each configured to perform the actions of the methods.

In another general aspect, in an embodiment, a crash cart includes a plurality of drawers. The crash cart also includes a removable user device docked to a surface of the crash cart, where the surface is above the plurality of drawers. The crash cart also includes an interior compartment under the surface of the crash cart and above the plurality of drawers, where the interior compartment is hidden from view when the removable user device is docked and is exposed when the removable user device is undocked. Other embodiments of this aspect include corresponding computer systems, apparatus, and computer programs recorded on one or more computer storage devices, each configured to perform various actions.

In another general aspect, in an embodiment, a non-transitory computer-program product includes a computer-usable medium having computer-readable program code embodied therein. The computer-readable program code is adapted to be executed to implement a method. The method includes receiving, by a user device, a code start trigger, where the user device is dockable on a crash cart and in communication with a controller disposed in relation to the crash cart. The method also includes, responsive to the code start trigger, initiating a code workflow, creating an event log for the code workflow, and determining a patient rhythm. The method also includes monitoring for real-time code events. The monitored real-time code events include patient intervention and a new patient rhythm. The method also includes, responsive to a determination that a real-time code event has occurred, executing programmed action that includes updating the event log.

### BRIEF DESCRIPTION OF THE DRAWINGS

A more complete understanding of the method and apparatus of the present disclosure may be obtained by reference to the following Detailed Description when taken in conjunction with the accompanying Drawings wherein:

FIG. 1A illustrates a front-right perspective view of a crash cart;

FIG. 1B illustrates a rear perspective view of a crash cart;

FIG. 1C illustrates a front-left perspective view of a crash cart;

FIG. 2A illustrates a removable user device relative to a dock;

FIG. 2B illustrates a dock for a removable user device;

FIG. 2C illustrates a view of a dock while a removable user device is docked thereto;

FIG. 2D illustrates a view of an underside of a dock while a removable user device 104 is docked thereto;

FIG. 2E illustrates a removable user device in a docked configuration;

FIG. 3 illustrates an operational view of a crash cart;

FIG. 4 illustrates an example of a computing environment for implementing a central management system;

FIG. 5 illustrates an example process for executing an emergency workflow on a crash cart;

FIG. 6A illustrates an example of a user interface for receiving at least a portion of an initial dataset;

FIG. 6B illustrates an example of a user interface for receiving at least a portion of an initial dataset;

FIG. 7 illustrates an example of a user interface that can be provided by during a code workflow;

FIG. 8 illustrates an example of a user interface that can be provided during a code workflow;

FIG. 9 illustrates an example of a user interface that can be provided during a code workflow.

FIG. 10 illustrates an example of a user interface that can be provided during a code workflow;

FIG. 11 illustrates an example of a user interface that can be provided for inventory tracking on a crash cart; and

FIG. 12 illustrates an example of a computer system.

### DETAILED DESCRIPTION

The following disclosure describes various illustrative embodiments and examples for implementing the features and functionality of the present disclosure. While particular components, arrangements, and/or features are described below in connection with various example embodiments, these are merely examples used to simplify the present disclosure and are not intended to be limiting. It will of course be appreciated that in the development of any actual embodiment, numerous implementation-specific decisions may be made to achieve the developer's specific goals, including compliance with system, business, and/or legal constraints, which may vary from one implementation to another. Moreover, it will be appreciated that, while such a development effort might be complex and time-consuming, it would nevertheless be a routine undertaking for those of ordinary skill in the art having the benefit of this disclosure.

While the making and using of various embodiments of the present disclosure are discussed in detail below, it should be appreciated that the present disclosure provides many applicable inventive concepts, which can be embodied in a wide variety of specific contexts. The specific embodiments discussed herein are merely illustrative and do not delimit the scope of the present disclosure. In the interest of clarity, not all features of an actual implementation may be described in the present disclosure.

In the Specification, reference may be made to the spatial relationships between various components and to the spatial orientation of various aspects of components as depicted in the attached drawings. However, as will be recognized by those skilled in the art after a complete reading of the present disclosure, the devices, components, members, apparatuses, etc. described herein may be positioned in any desired orientation. Thus, the use of terms such as “above”, “below”, “upper”, “lower”, “top”, “bottom” or other similar terms to describe a spatial relationship between various components or to describe the spatial orientation of aspects of such components, should be understood to describe a relative relationship between the components or a spatial orientation of aspects of such components, respectively, as the components described herein may be oriented in any desired direction. When used to describe a range of dimensions or other characteristics (e.g., time, pressure, temperature) of an element, operations, and/or conditions, the phrase “between X and Y” represents a range that includes X and Y.

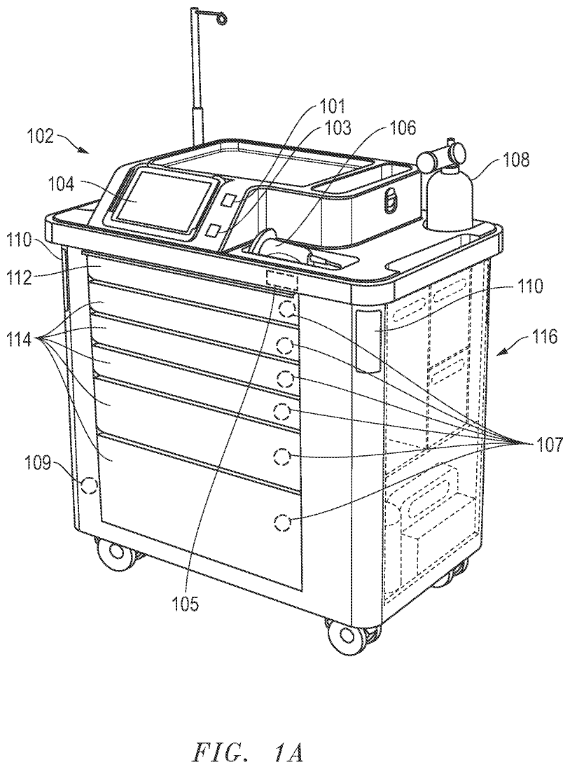

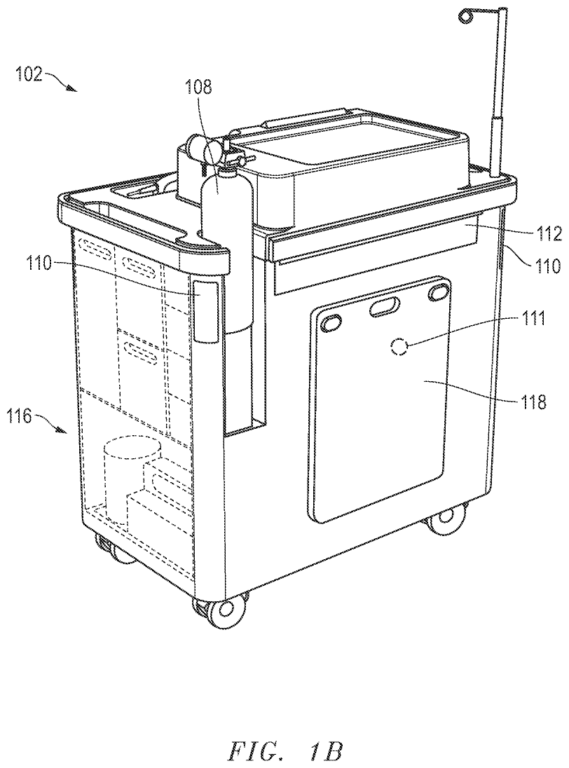

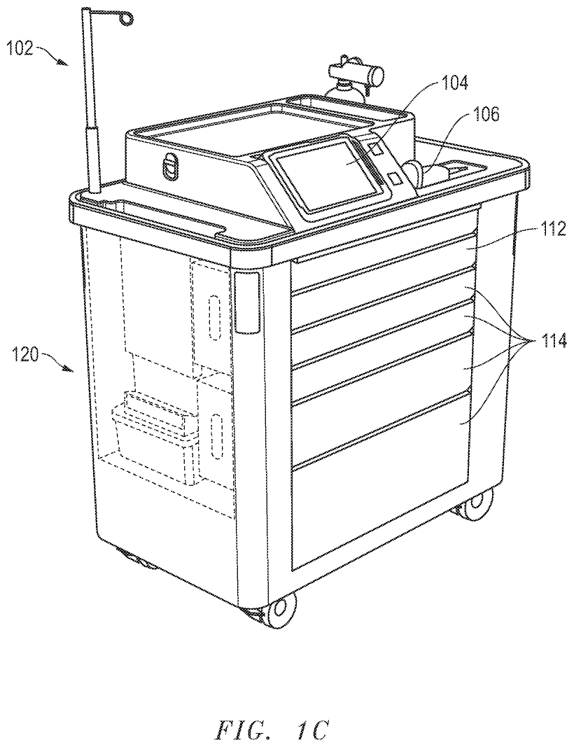

FIGS. 1A-C illustrates an example of a crash cart 102. FIGS. 1A, 1B and 1C illustrate front-right, rear, and front-left perspective views of the crash cart 102, respectively. As best seen in FIGS. 1A and 1B, the crash cart 102 includes a storage area 116 on a right side thereof. The storage area 116 can include, for example, transparent tilt-out bins. As best seen in FIG. 1C, the crash cart 102 includes a storage area 120 on a left side thereof. The storage area 120 can include, for example, mounted glove boxes, a sharps container, backup paper documentation, combinations of the foregoing and/or the like. It should be appreciated that the storage area 116 and the storage area 120 can be configured in any suitable fashion for a given crash-cart implementation.

The crash cart 102 includes a two-way drawer 112 and a plurality of one-way drawers 114. As shown in FIGS. 1A and 1B, the two-way drawer 112 is accessible from either a front or rear of the crash cart 102 and is configured to open from either direction. The two-way drawer 112 and the plurality of one-way drawers 114 can each include drugs, supplies or the like that may be needed in a medical emergency. Although the crash cart 102 is depicted, by way of example, as including particular quantities of one-way and two-way drawers, it should be appreciated that various implementations can include any suitable quantity of one-way and two-way drawers.

Still with reference to FIGS. 1A-C, the crash cart 102 is shown to include a removable user device 104, a radio frequency identification (RFID) sensor 101, a biometric sensor 103, and a barcode scanner 106 on top surface thereof. The removable user device 104 can be a computer such as, for example, a tablet or laptop, that is operable to dock and undock from the crash cart 102. In the illustrated embodiment, the removable user device 104 is depicted as a tablet computer for illustrative purposes. In various embodiments, the removable user device 104 is a user-facing device that provides a user interface for cart operations. In various embodiments, the removable user device 104 is communicably coupled to the RFID sensor 101 and the biometric sensor 103 via, for example, a dock, so that the a user can login to the removable user device 104 via either the RFID sensor or the biometric sensor 103. The biometric sensor 103 can be, for example, a fingerprint sensor.

Still with reference to FIGS. 1A-C, an oxygen tank 108 and a cardiac board 118 are disposed in designated locations on the rear of the crash cart 102, as best seen in FIG. 1B. Indicator lights 110 (e.g., light-emitting diodes (LEDs)) are disposed on each of four corners of the crash cart 102, although it should be appreciated that the indicator lights 110 can exist in different locations and quantities than what is shown.

Still with reference to FIGS. 1A-C, several components are shown as being internal to the crash cart 102 by way of dashed lines. In particular, the crash cart 102 includes a cart controller 105, drawer sensors 107, a temperature and humidity sensor 109 and a cardiac-board sensor 111. The drawer sensors 107 are shown to include a sensor for each of the two-way drawer 112 and the plurality of one-way drawers 114. The drawer sensors 107 be disposed in any suitable location in relation to such drawers. In a typical embodiment, the drawer sensors 107 are operable to detect when the drawer with which it is associated is open or closed.

The temperature and humidity sensor 109 is operable to detect, for example, temperature, relative humidity, and/or other environmental factors. The cardiac-board sensor 11 is operable to detect, for example, whether the cardiac board is present or absent from its designated location on the crash cart 102. It should be appreciated that the temperature and humidity sensor 109 can be located in any location in or on the crash cart 102. In similar fashion, in some cases, the crash cart 102 may have more than of the temperature and humidity sensor 109.

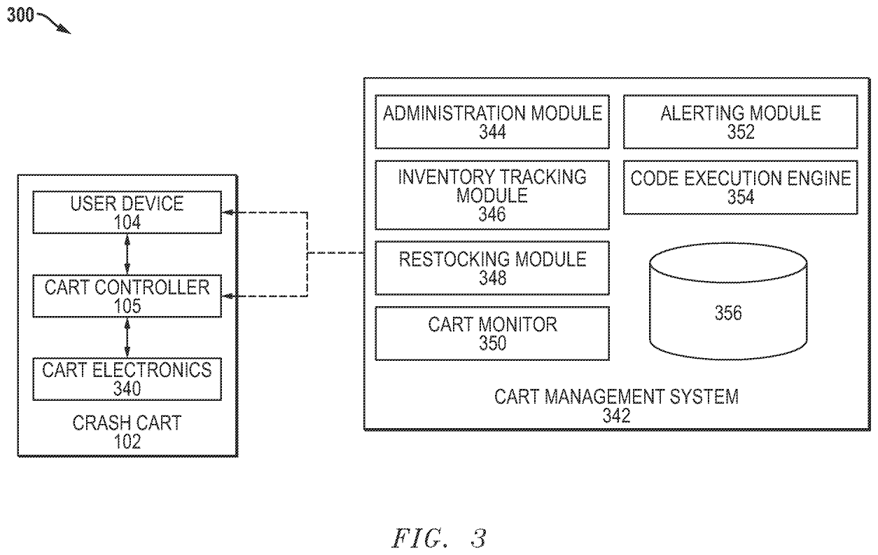

In a typical embodiment, the cart controller 105 controls operation of the crash cart 102. The cart controller 105 can be, for example, a computer that is disposed in any suitable location in or on the crash cart 102. In a typical embodiment, the cart controller 105 is communicably coupled to, and operable to control and/or receive information from, each of the cart electronics discussed above. In various cases, the cart controller 105 can be communicably coupled to such components via wired or wireless methods. For example, the cart controller 105 can be communicably coupled to the RFID sensor 101, the biometric sensor 103, the removable user device 104, the barcode scanner 106, the drawer sensors 107, the temperature and humidity sensor 109, the indicator lights 110 and/or the cardiac-board sensor 111. Operation of the cart controller 105 will be described in greater detail relative to FIG. 3.

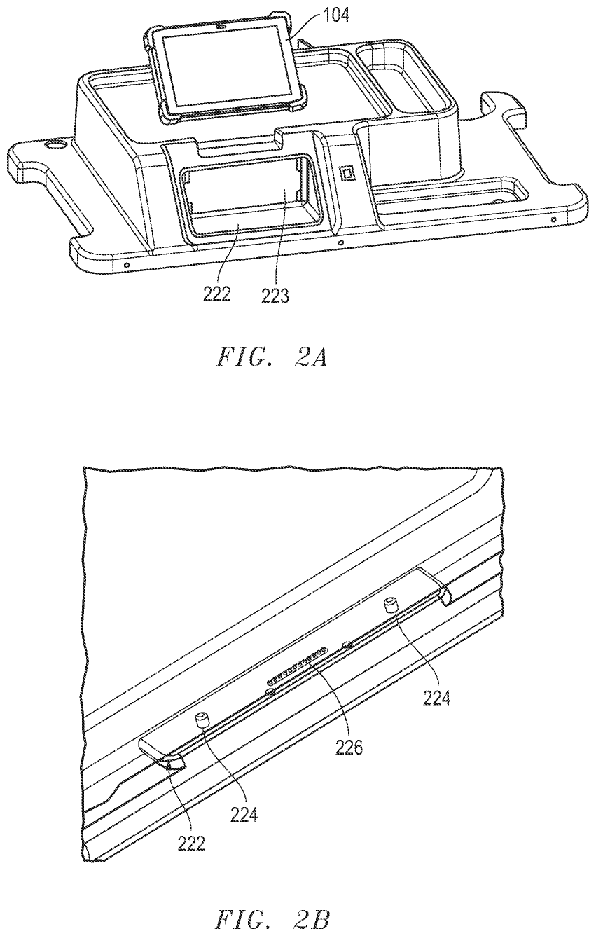

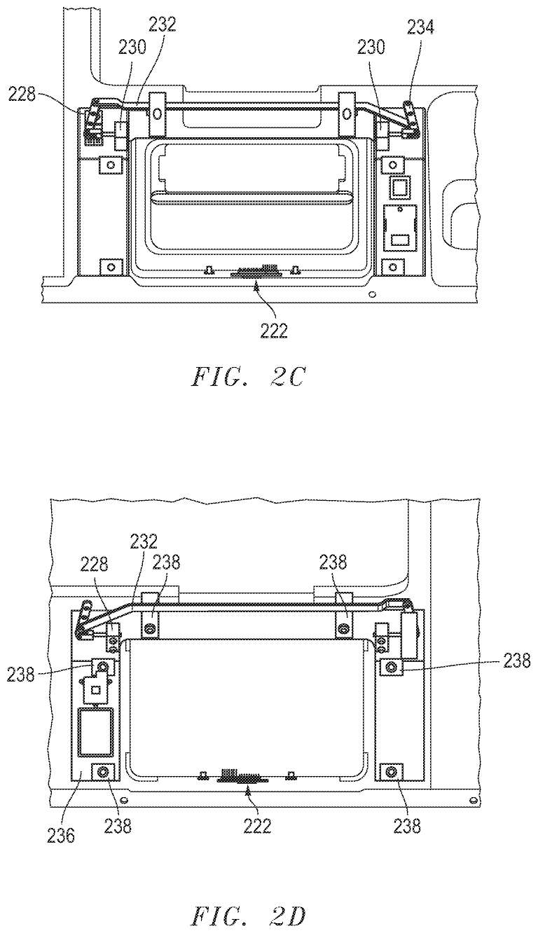

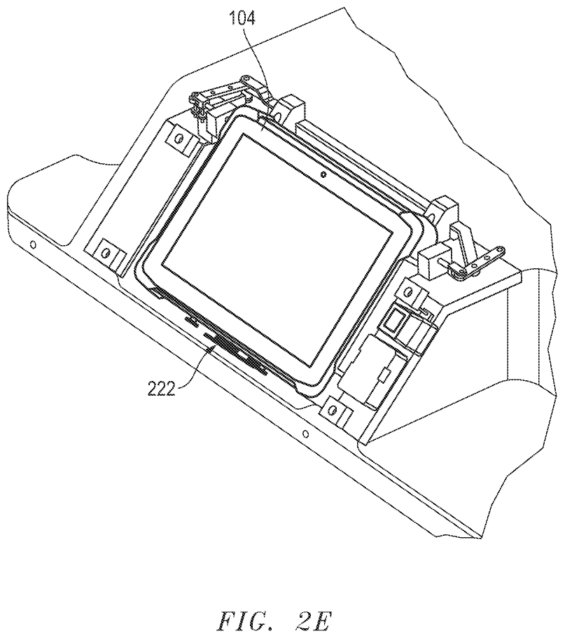

FIGS. 2A-C illustrate removability features related to the removable user device 104. FIG. 2A illustrates that the removable user device 104 is can be removed, or undocked, from a dock 222 to expose a hidden interior compartment 223. In various embodiments, the hidden interior compartment 223 can be used to store high-risk and/or high-value items such as controlled substances. In various embodiments, the removable user device 104 covers or hides the hidden interior compartment 223 from view when the removable user device 104 is docked.

FIG. 2B illustrates the dock 222 for the removable user device 104. The dock 222 is shown to include passive pins 224 on opposite ends thereof and a series of contacts 226, sometimes referred to as “pogo pins.” The passive pins 224 locate the removable user device 104 on the dock 222, while the dock 222 facilitates electrical communication between the dock 222 and the removable user device, for example, for purposes of powering the removable user device 104 and/or charging a battery of removable user device 104.

FIG. 2C illustrates a view of the dock 222 while the removable user device 104 is docked thereto. In the view of FIG. 2C, exterior casing is removed to better show interior components. In a typical embodiment, docking of the removable user device 104 to the dock 222, such that the removable user device 104 makes electrical contact with the series of contacts 226, activates a servo motor 228. The servo motor 228 thereafter moves locking pins 230 into corresponding holes on each side of the removable user device 104. In a typical embodiment, motion of the locking pins 230 is controlled by a linkage system 232. The linkage system 232 provides an additional lever arm and a mounting hole 234 for a manual override in the event that the servo motor 228 fails to unlock the removable user device 104. In a typical embodiment, actuating the linkage system 232 via the mounting hole 234 performs the same unlocking action as would the servo motor 228.

FIG. 2D illustrates a view of an underside of the dock 222 while the removable user device 104 is docked thereto. In the view of FIG. 2D, exterior casing is again removed to better show interior components. Locking mechanisms such as the servo motor 228 and the linkage system 232 are shown mounted to a sheet-metal structure 236 via couplers 238 having threaded inserts. The couplers 238 can be, for example, “glue boxes.” In various embodiments, the sheet-metal structure 236 alleviates higher tolerances that may be required by a thermoformed top surface and facilitates assembly.

FIG. 2E illustrates the removable user device 104 docked in the dock 222 according to the mechanisms described in FIGS. 2A-D.

FIG. 3 illustrates an operational view 300 of the crash cart 102. The operational view 300 includes a cart management system 342 deployed on the crash cart 102. The crash cart 102 includes the removable user device 104, the cart controller 105, and cart electronics 340. The cart controller 105 may engage in bidirectional wired and/or wireless communication with both the removable user device 104 and various components in the cart electronics 340. The cart management system 342 includes an administration module 344, an inventory tracking module 346, a restocking module 348, a cart monitor 350, an alerting module 352, a code execution engine 354, and memory 356.

In various embodiments, the cart management system 342 can be deployed on the cart controller 105, on the removable user device 104, or on a combination thereof such that the cart management system 342 and/or modules thereof represent distributed applications. In embodiments in which the cart controller 105 is deployed solely on the removable user device 104, the removable user device 104 may also serve as the cart controller 105, such that the cart controller 105 is not a physically distinct component in these embodiments. For illustrative purposes, the cart management system 342 will be described in a distributed fashion, with the removable user device 104 handling user-interface functionality and most other activity being allocated to the cart controller 105.

With particular reference to the crash cart 102, the cart electronics 340 can include sensors, indicators, and other electronics within the crash cart 102. For example, with reference to FIGS. 1A-C the cart electronics 340 can logically represent the RFID sensor 101, the biometric sensor 103, the removable user device 104, the barcode scanner 106, the drawer sensors 107, the temperature and humidity sensor 109, the indicator lights 110 and/or the cardiac-board sensor 111. The cart controller 105, via the cart management system 342, can monitor and/or control operation of the cart electronics 340.

The administration module 344 facilitates administration and setup of the crash cart 102 For example, the administration module 344 can receive or establish configuration settings for the crash cart 102 and store the configuration settings in the memory 356. The configuration settings can include, for example, tray configurations for each of the two-way drawer 112 and the plurality of one-way drawers 114, where each drawer includes or is organized as a tray having multiple compartments. For a given drawer, a tray configuration can identify a tray layout and indicate medications or supplies that are to be maintained in each compartment, optionally including required minimum quantities and/or maximum quantities of the same. Further, in various embodiments, the configuration settings can include inventory tracking settings for the crash cart 102. The inventory tracking settings can specify various parameters such as, for example, how often an inventory of the crash cart 102 is checked or updated.

In another example, the configuration settings received or established by the administration module 344 can include monitoring settings for the crash cart 102. The monitoring settings can specify, for example, thresholds or triggers for alerting in a specified fashion (e.g., when to alert, how to alert, whom to alert, etc.) In an example, the monitoring settings can include temperature or humidity thresholds, for example, for measurements determined by the temperature and humidity sensor 109. In some cases, such temperature or humidity thresholds may be established in conformity to standards articulated by a manufacturer of a given medication or supply in the crash cart 102.

In still another example, the configuration settings received or established by the administration module 344 can include emergency workflow settings for particular emergency scenarios, such as for what is commonly known as a “code blue.” In the example of a code blue, code workflow settings can be received or established that specify, for example, one or more trigger events for the code (e.g., a user indication). In addition, or alternatively, the code workflow settings can establish workflow activities as a function of a patient's cardiac rhythm. For example, the code workflow settings can specify a plurality of cardiac rhythms such as, for example, atrial fibrillation (AFIB), unstable bradycardia (BRADY), asystole (ASYS), pulseless electrical activity (PEA), ventricular fibrillation (VFIB), ventricular tachycardia (VTACH), and supraventricular tachycardia (SVT), or the like. For each specified cardiac rhythm, the code workflow settings can specify, for example, one or more patient interventions, some of which may be timed interventions associated with preconfigured time intervals for completing and/or repeating the intervention. The interventions may be organized into intervention categories such as airway management, vascular access, medications, or the like.

The inventory tracking module 346, in combination with the restocking module 348, can maintain a current inventory state of the crash cart 102 in the memory 356. The current inventory state can include, for example, the contents of the two-way drawer 112 and the plurality of one-way drawers 114 relative to the tray settings described above. The current inventory state can be maintained, for example, on an item-by-item basis, drawer-by-drawer basis, and/or compartment-by-compartment basis, so that it can be indicated, potentially graphically, which items are present and which items are missing on a configurably granular level.

In a typical embodiment, the inventory tracking module 346 can track medications and other supplies that may have been used, for example, during an emergency situation such as a code workflow. In certain embodiments, a user can login to the removable user device 104, enter the inventory tracking module 346, and scan each item in the two-way drawer 112 and the plurality of one-way drawers 114 using the barcode scanner 106. When the user has finished scanning, the current inventory state is defined by the items that have been scanned. The difference or delta between the last inventory state and the current inventory state (i.e., items indicated by the last inventory state but that were not scanned in the most recent check) represents items used, for example, during a most recent code workflow. In various embodiments, this difference can be recorded in the memory 356, for example, for billing or cost recovery. The difference between the current inventory state and items indicated, for example, by the tray settings, represents overall missing items. In many cases, these two differences will be the same. As mentioned previously, used and/or missing items can be maintained on an item-by-item basis, drawer-by-drawer basis, and/or compartment-by-compartment basis, so that it can be indicated, potentially graphically, which items are present and which items are missing on a configurably granular level.

The restocking module 348 can update the current inventory state of the crash cart 102, for example, as part of a restocking process. In various cases, the restocking module 348 can access the missing or used items in the memory 356 and indicate (e.g., graphically) the same to the user. In certain embodiments, the user can login to the removable user device 104, enter the restocking module 348, review the missing or used items, and scan in replacement items using the barcode scanner 106. When the user has finished scanning, the scanned items are added to the current inventory state maintained in the memory 356. In some embodiments, all historical inventory states are maintained in the memory 356 for auditing purposes.

The cart monitor 350 can monitor the cart electronics 340 in accordance with the monitoring settings and/or other settings. For example, the cart monitor 350 can monitor and track docking and undocking of the removable user device 104 via the dock 222, presence or absence of the cardiac board 118 via the cardiac-board sensor 111, opening and closing of the two-way drawer 112 and the plurality of one-way drawers via the drawer sensors 107, and the like. In other examples, the cart monitor 350 can monitor the current inventory state and/or whether missing or used items have been indicated by the inventory tracking module 346. Each of the aforementioned conditions and/or other conditions can be specified in the monitoring settings as triggers for configurable alerting. For example, the fact that the current inventory state indicates missing items may trigger alerting. In another example, the fact that the current inventor state has not been checked for a configurable period of time may trigger alerting.

In some cases, triggers can specify a time duration of the condition before any alerting takes place (e.g., drawer open for at least thirty seconds). In addition, or alternatively, the cart monitor 350 can record data or information collected from the foregoing components and/or other components in the memory 356. In some embodiments, the cart monitor 350 can trigger non-alerting operations, such as another module of the cart management system 342. For example, upon a trigger event for an emergency or code situation, the cart monitor 350 can initiate the code execution engine 354. Other examples will be apparent to one skilled in the art after a detailed review of the present disclosure.

The alerting module 352 is operable to initiate alerting according to various mechanisms and protocols. For example, in an embodiment, the alerting module 352 can issue alerts to a configurable user or group of users via text message, email, phone call, enterprise messaging, or the like. In another example, in an embodiment, the alerting module 352 can issue audible alerts via a speaker coupled to the crash cart 102. In still another example, the alerting module 352 can initiate visible alerts by causing the indicator lights 110 to become lit or to flash in a suitable pattern indicative of a given alert. In yet another example, the alerting module 352 can cause audible and/or visible alarms throughout a facility to be triggered. Other examples of alerting methods and mechanisms will be apparent to one skilled in the art after a detailed review of the present disclosure.

The code execution engine 354 can execute an emergency workflow, including but not limited to what is commonly known as a “code blue.” The code execution engine 354 can operate utilizing the code workflow settings described above. When a code has been triggered, the code execution engine 354 initiates a code workflow and creates a new code event log. During the code workflow, the code execution engine 354 monitors for real-time code events such as, for example, new information related to patient rhythm, patient interventions and patient status, and takes configurable and programmed action based thereon. The programmed action can include, for example, recording the event in the event log in relation to the time at which it occurred. If the code event is a change in patient rhythm, the programmed action can further include, for example, updating a list of interventions presented to the user in correspondence to the code workflow settings discussed above, and starting timers for any interventions that are timed. If the code event is the completion of a timed intervention that is to be repeated, the programmed action can further include restarting the timer associated with the timed intervention. Throughout the code workflow, the code execution engine 354 can visually indicate real-time countdowns for each timer to a user of the removable user device 104.

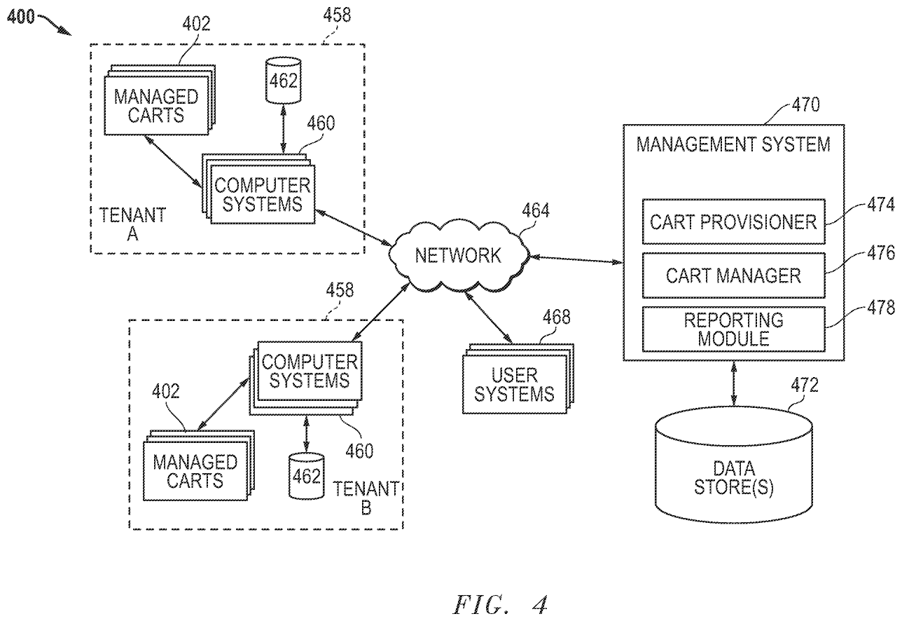

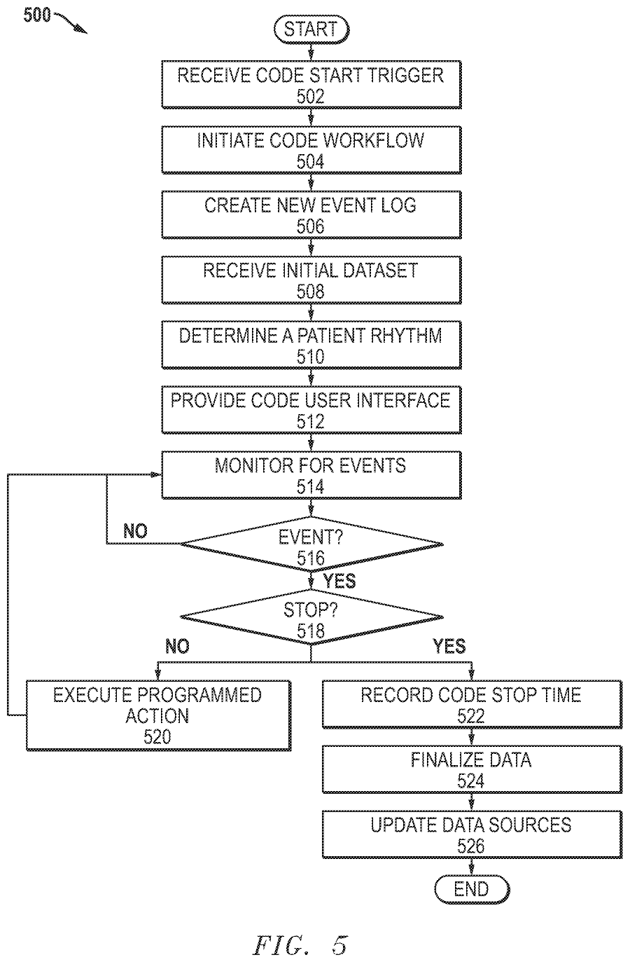

Upon conclusion of the code workflow (e.g., upon a user-indicated code-stop event), the code execution engine 354 can store the event log in the memory 356. In some embodiments, the user will be given an opportunity to edit, correct, or supplement the event log. In some embodiments, the event log and/or other data related to the code workflow is transmitted to an external system for storage with a patient record. Example operation of the code execution engine 354 will be described in greater detail relative to FIG. 4.

FIG. 4 illustrates an example of a computing environment 400 for implementing a central management system 470 that can enable automation and tracking of crash carts across the same or multiple sites. The computing environment 400 includes the central management system 470, tenant systems 458, user systems 468 and one or more data stores 472, each of which is operable to communicate over a network 464. The network 464 may be, or include, one or more of a private network, a public network, a local or wide area network, a portion of the Internet, combinations of the same, and/or the like.

In certain embodiments, the central management system 470 can centrally manage crash-cart deployments for its tenants, each of which may correspond to a hospital, hospital system, or medical facility in some embodiments. In particular, in the computing environment 400, the tenant systems 458 can be served by the central management system 470. In general, the tenant systems 458 can each be considered an abstraction of actual crash-cart deployments managed by the central management system 470 and the systems and data sources with which those crash-cart deployments interact. For example, one of the tenant systems 458 is shown as owned or operated by “Tenant A” while another system 458 is owned or operated by a different tenant, “Tenant B.” The tenant systems 458 shown can be owned or operated by the same or different entities. For example, Tenants A and B can represent customers (e.g., entities such as companies or individuals) of an operator of the central management system 470. Although the term “tenant” is used herein to describe the tenant systems 458 or owners/operators thereof, in addition to having its ordinary meaning, the term “tenant” can, but need not, refer to tenancy in a multitenant software architecture.

The tenant systems 458 are each shown to include one or more managed carts 402, one or more computer systems 460 and one or more data sources 462. The managed carts 402 can each operate as described, for example, with respect to the crash cart 102 of FIGS. 1A-C, 2A-E and 3. The one or more computer systems 460 can each be, for example, a computer system for a hospital, hospital system or other medical facility. The one or more data sources 462 can include data streams or datasets that can be received or processed by the computer systems 460, for example, from the managed carts 402 (e.g., any data that may be stored in the memory 356 of FIG. 3). In various cases, the one or more data sources 462 can be updated by the managed carts 402, by the computer systems 460, or other components, in real-time, on a periodic basis, e.g., according to a schedule, on-demand or a combination of the same.

In the illustrated embodiment, the central management system 470 can include a cart provisioner 474, a cart manager 476, and a reporting module 478. Each of these components can be implemented with hardware and/or software, including (optionally) virtual machines. In an example, the central management system 470 can be implemented as a single management server. In another example, the central management system 470 can be implemented in a plurality of virtual or physical servers, which may or may not be geographically co-located. In some embodiments, the central management system 470 and/or other aspects of the computing environment 400 may be hosted on a cloud system.

In certain embodiments, features of the components of the central management system 470 can be made accessible over an interface to the user systems 468. The user systems 468 can include any type of computing device, including information handling systems such as desktops, laptops, tablets, smartphones, and wearable or body-borne computers, to name a few. The user systems 468 can be operated by users, such as human users, associated with the tenants or by other users.

The cart provisioner 474 can be utilized to create and provision a crash cart similar to the crash cart 102 with a cart management system and/or configuration settings for a particular type of cart, such that the crash cart becomes one of the managed carts 402. In some embodiments, the cart management system and the configuration settings are created or retrieved, for example, from the data store(s) 472. The cart management system can be similar, for example, to the cart management system 342 of FIG. 3. The configuration settings can be similar to the configurations settings described above relative to FIG. 3. In various embodiments, the cart management system and/or the configuration settings can vary by tenant and/or type of cart.

In some embodiments, the cart provisioner 474 includes or provides a configuration interface for creating and/or provisioning crash carts. The configuration interface can be accessible, for example, by the user systems 468, and can be tenant-specific for particular tenants. In certain embodiments, the cart provisioner 474 can be used to provision a single cart or a plurality of carts concurrently. In certain embodiments, the cart provisioner 474 uses and maintains in the data store(s) 472, for each of the tenant systems 458, provisioning settings indicative of specific locations or paths where some or all configuration settings for its carts reside and/or specific interfaces for retrieving some or all of the configuration settings. The provisioning settings can further include, for example, connectivity information for one or more of the computer systems 460 and/or data stores with which a given cart may interact to execute its functionality.

The cart manager 476 can serve to manage the managed carts 402 for tenants. In certain embodiments, the cart manager 476 can issue commands to control operation of bots. The cart manager 476 can be utilized to re-configure, optimize and/or customize any of the managed carts 402. For example, various commands can activate or deactivate carts, perform configuration management similar to the above-described provisioning of configuration settings, combinations of the same and/or the like. In some cases, the cart manager 476 can publish a configuration interface to the user systems 468, for example, for administrators, super users or other users (e.g., of a particular tenant) to select or specify such commands.

The reporting module 478 can generate regular or on-demand reports related to the managed carts 402. In various cases, these reports can provide a snapshot of some or all of the managed carts 402. The reporting module 478 can publish reports or other generated information, for example, to a web, and/or the like. The reporting module 478 can generate and execute a page, dashboard, and/or query of the data store(s) 472. The web page, user dashboard or other user interface(s) output, for example, by the reporting module 478, can be accessed by users of the user systems 468.

In general, the data store(s) 472 can include any information collected, stored or used by the central management system 470. For example, in various embodiments, the data store(s) 472 can include configuration settings, provisioning settings, data collected from the managed carts 402, data received or collected from the user systems 468, combinations of the same and/or the like. In certain embodiments, data stored in the data store(s) 472 can take the form of repositories, flat files, databases, etc. In certain embodiments, the data store(s) 472 can be utilized as an event library including event logs for the managed carts 402, in which actions performed by any of the managed carts 402 are stored, for example, by tenant.

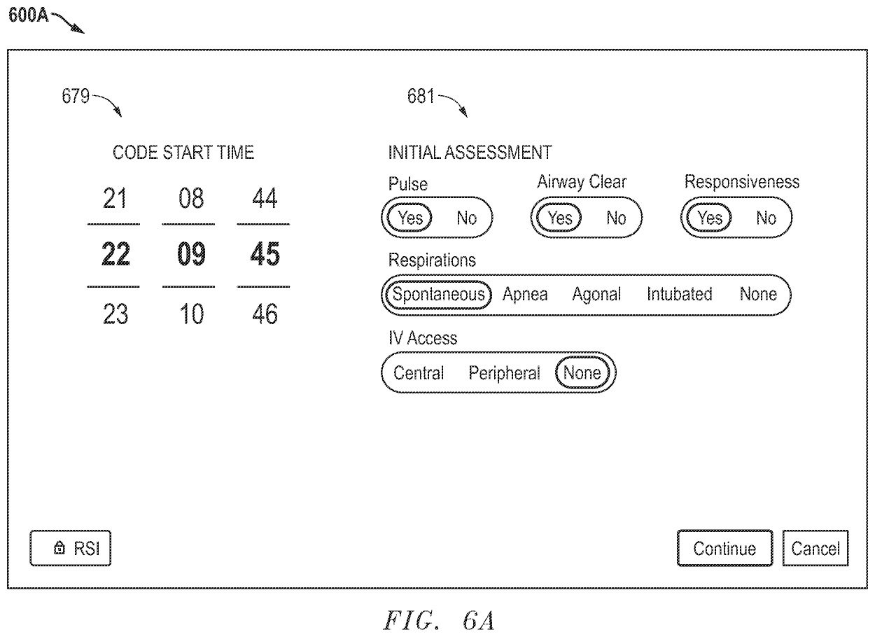

FIG. 5 illustrates an example process 500 for executing an emergency workflow such as a code workflow on a crash cart. In various embodiments, the process 500 can be executed by the crash cart 102 of FIGS. 1A-C, 2A-E and 3 and/or one of the managed carts 402 of FIG. 4. In addition, or alternatively, the process 500 can be executed by the cart management system 342 and/or the code execution engine 354 of FIG. 3. Although any number of components or systems may execute the process 500 in various implementations, for simplicity of description, the process 500 will be described relative to the code execution engine 354 of FIG. 3.

At block 502, the code execution engine 354 receives a code start trigger. In an example, the code start trigger can be received, for example, from a user via a user interface on the removable user device 104. In other examples, the code start trigger can be received via a button press on the crash cart 102. In some cases, the code start trigger can be received remotely.

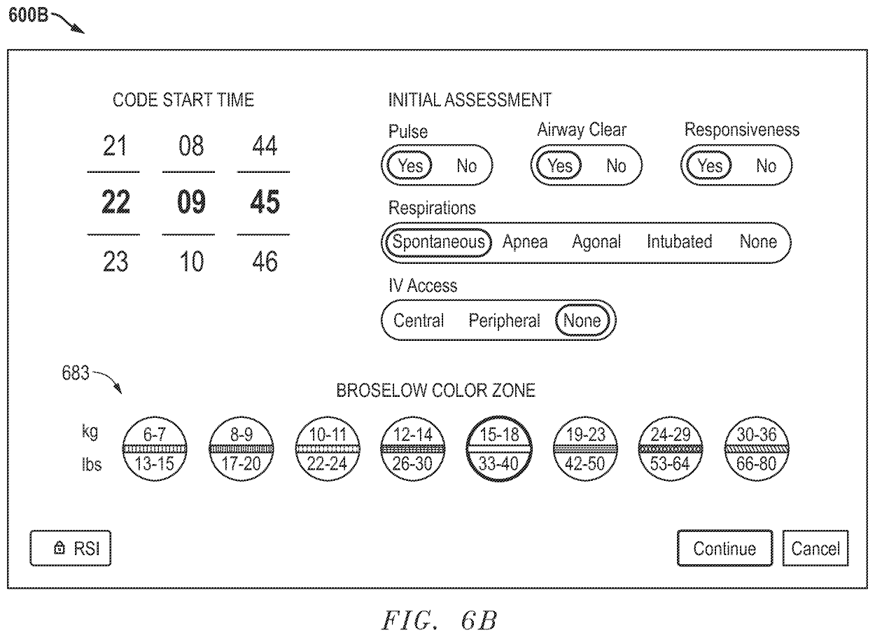

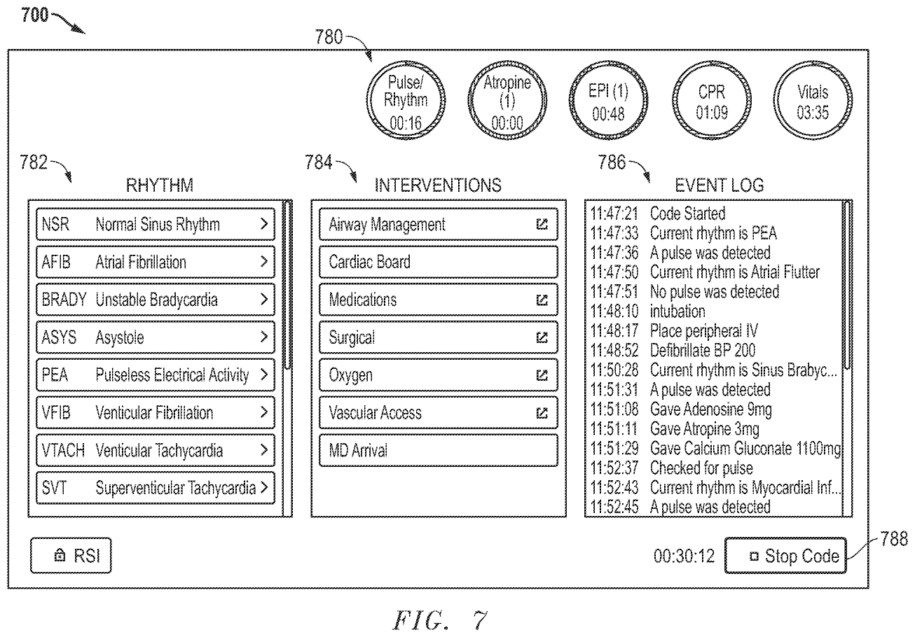

At block 504, the code execution engine 354 initiates a code workflow, for example, by serving a code user interface to the removable user device 104. Examples of the code user interface will be shown and described relative to FIGS. 6A-B and 7-10. At block 506, the code execution engine 354 creates a new event log for the code workflow.

At block 508, the code execution engine 354 receives an initial dataset for the code workflow. The initial dataset can include, for example, an initial assessment of a patient that is the subject of the code workflow, a code start time, and/or other data. In some embodiments, the code start time is pre-populated with a time of the code start trigger, so that the user can modify the code start time if, for example, the code should actually be registered at an earlier time. In some embodiments, the block 508 can be omitted or performed at the end of the code workflow if, for example, urgency so dictates.

At block 510, the code execution engine 354 determines a patient rhythm such as, for example, NSR, AFIB, BRADY, ASYS, PEA, VFIB, VTACH, SVT, or the like. In certain embodiments, the patient rhythm can be user-indicated via the removable user device 104 or included in the initial dataset. In some embodiments, the patient rhythm can be determined automatically via communication with medical sensors.

At block 512, the code execution engine 354 provides a code user interface. The code user interface can include, for example, a listing of interventions associated with the patient rhythm, a real-time view of the event log, and/or other information. In some cases, the interventions associated with the patient rhythm may be timed interventions that occur or that are repeated upon the expiration of a time interval. In these cases, the code user interface can further include, for example, visual timers that graphically countdown the time intervals and audibly and/or visually prompt the user upon the expiration thereof. As described previously, the intervention associations and the time intervals can be specified in the configuration settings in the memory 356. An example of a code user interface will be described relative to FIGS. 6A-B and 7-10.

At block 514, the code execution engine 354 monitors for configurable real-time code events such, for example, new information related to patient rhythm, patient interventions, and patient status. At decision block 516, the code execution engine 354 determines whether a configurable real-time code event has occurred. If not, the process 500 returns to the block 514 and executes as described previously. Otherwise, if it is determined at the decision block 516 that a configurable code event has occurred, the process 500 proceeds to decision block 518.

At decision block 518, the code execution engine 354 determines whether the real-time code event is a stop code event. If not, at block 520, the code execution engine 354 executes configurable programmed action based on the real-time code event. The programmed action can vary with the type of the real-time code event. In most cases, the programmed action can include, for example, recording the event in the event log in relation to the time at which it occurred. If the code event is a new patient rhythm, the programmed action can further include, for example, updating the code user interface to include the interventions associated with the new patient rhythm and starting timers for any interventions associated therewith that are timed. If the code event is the completion of a timed intervention that is to be repeated, the programmed action can further include restarting the timer associated with the timed intervention. From block 520, the process 500 returns to the block 514 and executes as described previously.

If it is determined at the decision block 518 that the real-time code event is a stop-code event, at block 522, the code execution engine 354 records a code stop time in correspondence to a time associated with the stop-code event (e.g., a time of receipt). At block 524, the user of the removable user device 104 is prompted finalize data related to the code work flow. The block 524 can include, for example, providing an interface on the removable user device 104 for the user to correct or update the event log and/or other data associated with the code workflow. In addition, or alternatively, the block 524 can include prompting the user for, and receiving, additional data about the code workflow such as, for example, parties present or any other missing information.

At block 526, the code execution engine 354 updates applicable data sources with data resulting from the code workflow. Such data, including the event log, can be stored in the memory 356, stored in a data store such as one of the data sources 462 of FIG. 4, transmitted to a tenant system such as one of the computer systems 460 of FIG. 4 for storage, transmitted to a central management system such as the central management system 470 of FIG. 4 for storage in the data store(s) 472, or otherwise suitably handled. In addition, or alternatively, some or all of such data can be sent to a separate healthcare or medical system for storage as part of a patient record. After block 526, the process 500 ends.

FIG. 6A illustrates an example of a user interface 600A for receiving at least a portion of an initial dataset as described, for example, relative to the block 508 of FIG. 5. The user interface 600A can be provided, for example, on the removable user device 104 of FIGS. 1A-C, 2A-E and 3. The user interface 600A includes an area 679 for receiving a code start time, where the area 679 is pre-populated with a time associated with a code start trigger as described above relative to the block 508 of FIG. 5. The user interface 600A also includes an area 681 for receiving data related to an initial assessment of the patient.

FIG. 6B illustrates an example of a user interface 600B for receiving at least a portion of an initial dataset as described, for example, relative to the block 508 of FIG. 5. The user interface 600B can be provided, for example, on the removable user device 104 of FIGS. 1A-C, 2A-E and 3. The user interface 600B is similar to the user interface 600A of FIG. 6A and further includes an area 683 for receiving information related to a Broselow color zone. In various embodiments, the user interface 600B may be suitable for receiving an initial dataset for a pediatric patient.

FIG. 7 illustrates an example of a user interface 700 that can be provided by the code execution engine 354 during a code workflow. The user interface 700 can be provided, for example, on the removable user device 104 of FIGS. 1A-C, 2A-E and 3. The user interface 700 can correspond to the code user interface as described relative to blocks 512 and 520 of FIG. 5.

The user interface 700 includes a timer area 780, a rhythm area 782, an intervention area 784, an event-log area 786, and a stop-code button 788. The timer area 780 includes a plurality of visual timers that graphically countdown time intervals for certain timed patient interventions. The rhythm area 782 can facilitate determination of a patient rhythm as described relative to the block 510 of FIG. 5 and/or a change to the patient rhythm as described relative to blocks 514-520 of FIG. 5. The intervention area 784 provides a listing of patient interventions and/or intervention categories and can facilitate entry of a completed intervention, for example, as a new real-time code event. The event-log area 786 presents a real-time event log that can be created and updated as described relative to FIG. 5. The stop-code button 788, when activated by a user, can trigger a stop-code event as described relative to the decision block 518 of FIG. 5.

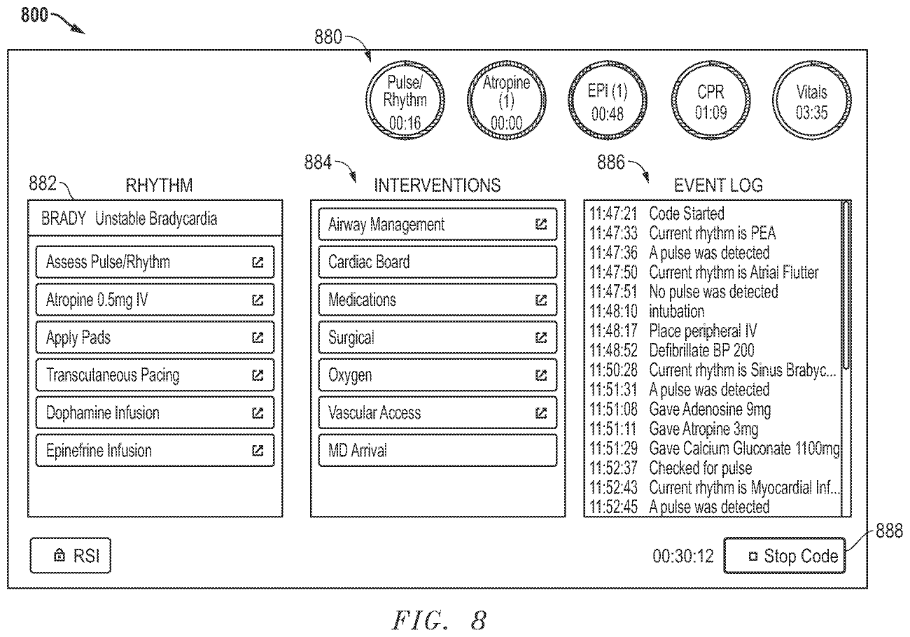

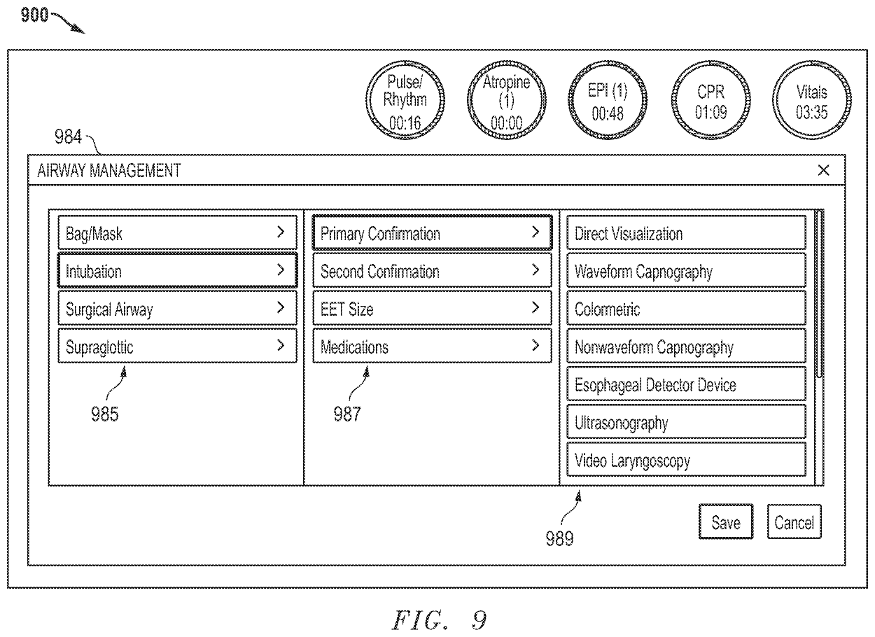

FIG. 8 illustrates an example of a user interface 800 that can be provided by the code execution engine 354 during a code workflow. The user interface 800 can be provided, for example, on the removable user device 104 of FIGS. 1A-C, 2A-E and 3. The user interface 800 can correspond to the code user interface as described relative to the blocks 512 and 520 of FIG. 5.

The user interface 800 includes a timer area 880, a rhythm area 882, an intervention area 884, an event-log area 886, and a stop-code button 888. The rhythm area 882 indicates a current patient rhythm of BRADY. The intervention area 884 provides a listing of patient interventions and/or intervention categories and can facilitate entry of a completed intervention, for example, as a new real-time code event. In some cases, the intervention area 884 can be filtered to only include categories and interventions that are associated with the patient rhythm BRADY, for example, according to configuration settings as described relative to FIG. 3. The timer area 880 includes a plurality of visual timers that graphically countdown time intervals for certain timed patient interventions that are associated with the patient rhythm BRADY. The stop-code button 888, when activated by a user, can trigger a stop-code event as described relative to the decision block 518 of FIG. 5.

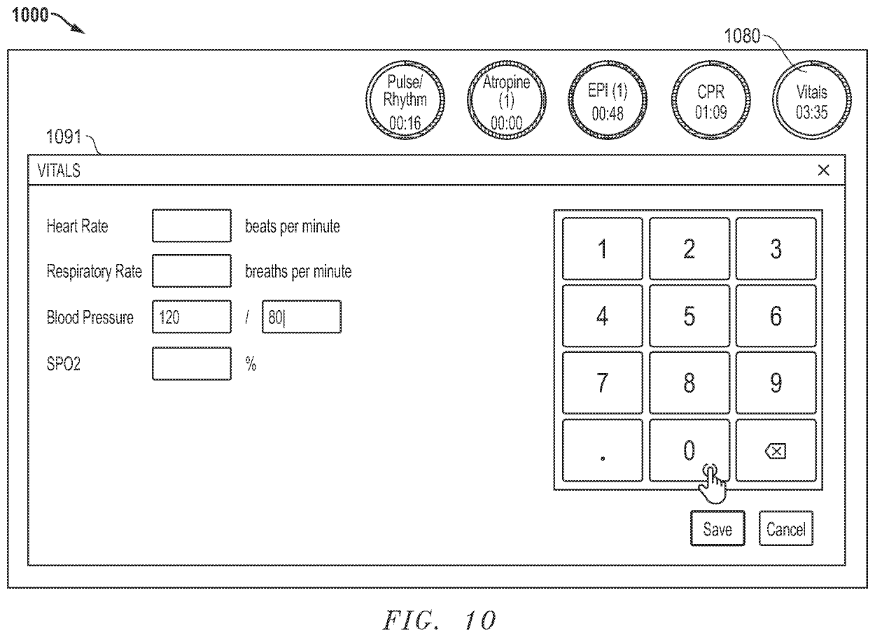

FIG. 9 illustrates an example of a user interface 900 that can be provided by the code execution engine 354 during a code workflow. The user interface 900 can be provided, for example, on the removable user device 104 of FIGS. 1A-C, 2A-E and 3. In an embodiment, the user interface 900 can be used to indicate a patient intervention that has been completed, where the completed patient intervention represents a new real-time code event as described relative to the process 500 of FIG. 5.

More particularly, the user interface 900 can represent a result of drilling down into the intervention area 884 of the user interface 800 shown in FIG. 8. The user interface 900 includes an intervention area 984 that further includes a first drill-down area 985, a second drill-down area 987, and a third drill-down area 989. The intervention area 984 indicates a category selection of “airway management.” The first drill-down area 985 then indicates a first subcategory selection of “intubation.” The second drill-down area 987 then indicates a second subcategory selection of “primary confirmation.” Finally, the third drill-down area 989 provides a listing of interventions for user selection, for example, as a new real-time code event.

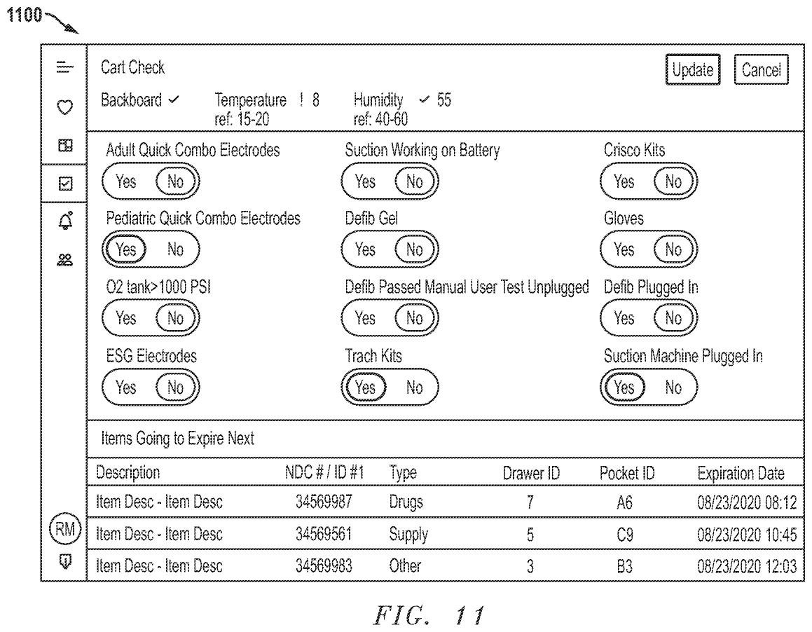

FIG. 10 illustrates an example of a user interface 1000 that can be provided by the code execution engine 354 during a code workflow. The user interface 1000 can be provided, for example, on the removable user device 104 of FIGS. 1A-C, 2A-E and 3. The user interface 1000 includes an area 1091 for entering patient vitals, entry of which corresponds to completion of a timed patient intervention corresponding to a visual timer 1080.

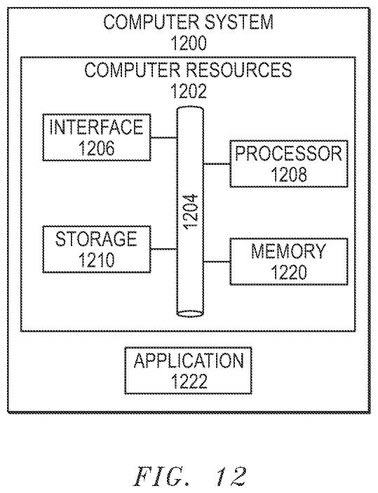

FIG. 11 illustrates an example of a user interface 1100 that can be provided by the inventory tracking module 346 of FIG. 3. The user interface 1100 can be provided, for example, on the removable user device 104 of FIGS. 1A-C, 2A-E and 3. The user interface 1100 facilitates a cart or inventory check as described above relative to FIG. 3.

FIG. 12 illustrates an example of a computer system 1200. In some cases, the computer system 1200 can be representative, for example, of the removable user device 104 and/or the cart controller 105 of FIGS. 1A-C, 2A-E and 3. In addition, with reference to FIG. 4, the computer system 1200 can be representative of any of the tenant systems 458 or components thereof, the user systems 468, and/or the central management system 470 or components thereof. The computer system 1200 includes an application 1222 operable to execute on computer resources 1202. The application 1222 can include, for example, logic and processing described herein. In an example, the application 1222 can implement the cart management system 342 of FIG. 3, a module thereof, and/or other particular portions thereof. In particular embodiments, the computer system 1200 may perform one or more actions described or illustrated herein. In particular embodiments, one or more computer systems may provide functionality described or illustrated herein. In particular embodiments, encoded software running on one or more computer systems may perform one or more actions described or illustrated herein or provide functionality described or illustrated herein.

The components of the computer system 1200 may include any suitable physical form, configuration, number, type and/or layout. As an example, and not by way of limitation, the computer system 1200 may include an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a wearable or body-borne computer, a server, or a combination of two or more of these. Where appropriate, the computer system 1200 may include one or more computer systems; be unitary or distributed; span multiple locations; span multiple machines; or reside in a cloud, which may include one or more cloud components in one or more networks.

In the depicted embodiment, the computer system 1200 includes a processor 1208, memory 1220, storage 1210, interface 1206 and bus 1204. Although a particular computer system is depicted having a particular number of particular components in a particular arrangement, this disclosure contemplates any suitable computer system having any suitable number of any suitable components in any suitable arrangement.

Processor 1208 may be a microprocessor, controller, or any other suitable computing device, resource, or combination of hardware, software and/or encoded logic operable to execute, either alone or in conjunction with other components, (e.g., memory 1220), the application 1222. Such functionality may include providing various features discussed herein. In particular embodiments, processor 1208 may include hardware for executing instructions, such as those making up the application 1222. As an example, and not by way of limitation, to execute instructions, processor 1208 may retrieve (or fetch) instructions from an internal register, an internal cache, memory 1220, or storage 1210; decode and execute them; and then write one or more results to an internal register, an internal cache, memory 1220, or storage 1210.

In particular embodiments, processor 1208 may include one or more internal caches for data, instructions, or addresses. This disclosure contemplates processor 1208 including any suitable number of any suitable internal caches, where appropriate. As an example, and not by way of limitation, processor 1208 may include one or more instruction caches, one or more data caches and one or more translation lookaside buffers (TLBs). Instructions in the instruction caches may be copies of instructions in memory 1220 or storage 1210 and the instruction caches may speed up retrieval of those instructions by processor 1208. Data in the data caches may be copies of data in memory 1220 or storage 1210 for instructions executing at processor 1208 to operate on; the results of previous instructions executed at processor 1208 for access by subsequent instructions executing at processor 1208, or for writing to memory 1220, or storage 1210; or other suitable data. The data caches may speed up read or write operations by processor 1208. The TLBs may speed up virtual-address translations for processor 1208. In particular embodiments, processor 1208 may include one or more internal registers for data, instructions, or addresses. Depending on the embodiment, processor 1208 may include any suitable number of any suitable internal registers, where appropriate. Where appropriate, processor 1208 may include one or more arithmetic logic units (ALUs); be a multi-core processor; include one or more processors 1208; or any other suitable processor.

Memory 1220 may be any form of volatile or non-volatile memory including, without limitation, magnetic media, optical media, random access memory (RAM), read-only memory (ROM), flash memory, removable media, or any other suitable local or remote memory component or components. In particular embodiments, memory 1220 may include random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM, or any other suitable type of RAM or memory. Memory 1220 may include one or more memories 1220, where appropriate. Memory 1220 may store any suitable data or information utilized by the computer system 1200, including software embedded in a computer readable medium and/or encoded logic incorporated in hardware or otherwise stored (e.g., firmware). In particular embodiments, memory 1220 may include main memory for storing instructions for processor 1208 to execute or data for processor 1208 to operate on. In particular embodiments, one or more memory management units (MMUs) may reside between processor 1208 and memory 1220 and facilitate accesses to memory 1220 requested by processor 1208.

As an example, and not by way of limitation, the computer system 1200 may load instructions from storage 1210 or another source (such as, for example, another computer system) to memory 1220. Processor 1208 may then load the instructions from memory 1220 to an internal register or internal cache. To execute the instructions, processor 1208 may retrieve the instructions from the internal register or internal cache and decode them. During or after execution of the instructions, processor 1208 may write one or more results (which may be intermediate or final results) to the internal register or internal cache. Processor 1208 may then write one or more of those results to memory 1220. In particular embodiments, processor 1208 may execute only instructions in one or more internal registers or internal caches or in memory 1220 (as opposed to storage 1210 or elsewhere) and may operate only on data in one or more internal registers or internal caches or in memory 1220 (as opposed to storage 1210 or elsewhere).

In particular embodiments, storage 1210 may include mass storage for data or instructions. For example, in various embodiments, storage 1210 can store configurations such as the configurations 218 of FIG. 2. As an example, and not by way of limitation, storage 1210 may include a hard disk drive (HDD), a floppy disk drive, flash memory, an optical disc, a magneto-optical disc, magnetic tape, or a Universal Serial Bus (USB) drive or a combination of two or more of these. Storage 1210 may include removable or non-removable (or fixed) media, where appropriate. Storage 1210 may be internal or external to the computer system 1200, where appropriate. In particular embodiments, storage 1210 may be non-volatile, solid-state memory. In particular embodiments, storage 1210 may include read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. Storage 1210 may take any suitable physical form and may include any suitable number or type of storage. Storage 1210 may include one or more storage control units facilitating communication between processor 1208 and storage 1210, where appropriate.

In particular embodiments, interface 1206 may include hardware, encoded software, or both providing one or more interfaces for communication (such as, for example, packet-based communication) among any networks, any network devices and/or any other computer systems. As an example, and not by way of limitation, communication interface 1206 may include a network interface controller (NIC) or network adapter for communicating with an Ethernet or other wire-based network and/or a wireless NIC (WNIC) or wireless adapter for communicating with a wireless network.

Depending on the embodiment, interface 1206 may be any type of interface suitable for any type of network for which computer system 1200 is used. As an example, and not by way of limitation, computer system 1200 can include (or communicate with) an ad-hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 1200 can include (or communicate with) a wireless PAN (WPAN) (such as, for example, a BLUETOOTH WPAN), a WI-FI network, a WI-MAX network, an LTE network, an LTE-A network, a cellular telephone network (such as, for example, a Global System for Mobile Communications (GSM) network), or any other suitable wireless network or a combination of two or more of these. The computer system 1200 may include any suitable interface 1206 for any one or more of these networks, where appropriate.

In some embodiments, interface 1206 may include one or more interfaces for one or more I/O devices. One or more of these I/O devices may enable communication between a person and the computer system 1200. As an example, and not by way of limitation, an I/O device may include a keyboard, keypad, microphone, monitor, mouse, printer, scanner, speaker, still camera, stylus, tablet, touchscreen, trackball, video camera, another suitable I/O device or a combination of two or more of these. An I/O device may include one or more sensors. Particular embodiments may include any suitable type and/or number of I/O devices and any suitable type and/or number of interfaces 1206 for them. Where appropriate, interface 1206 may include one or more drivers enabling processor 1208 to drive one or more of these I/O devices. Interface 1206 may include one or more interfaces 1206, where appropriate.

Bus 1204 may include any combination of hardware, software embedded in a computer readable medium and/or encoded logic incorporated in hardware or otherwise stored (e.g., firmware) to couple components of the computer system 1200 to each other. As an example, and not by way of limitation, bus 1204 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCI-X) bus, a serial advanced technology attachment (SATA) bus, a Video Electronics Standards Association local (VLB) bus, or any other suitable bus or a combination of two or more of these. Bus 1204 may include any number, type and/or configuration of buses 1204, where appropriate. In particular embodiments, one or more buses 1204 (which may each include an address bus and a data bus) may couple processor 1208 to memory 1220. Bus 1204 may include one or more memory buses.

Herein, reference to a computer-readable storage medium encompasses one or more tangible computer-readable storage media possessing structures. As an example, and not by way of limitation, a computer-readable storage medium may include a semiconductor-based or other integrated circuit (IC) (such, as for example, a field-programmable gate array (FPGA) or an application-specific IC (ASIC)), a hard disk, an HDD, a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a SECURE DIGITAL card, a SECURE DIGITAL drive, a flash memory card, a flash memory drive, or any other suitable tangible computer-readable storage medium or a combination of two or more of these, where appropriate.

Particular embodiments may include one or more computer-readable storage media implementing any suitable storage. In particular embodiments, a computer-readable storage medium implements one or more portions of processor 808 (such as, for example, one or more internal registers or caches), one or more portions of memory 820, one or more portions of storage 810, or a combination of these, where appropriate. In particular embodiments, a computer-readable storage medium implements RAM or ROM. In particular embodiments, a computer-readable storage medium implements volatile or persistent memory. In particular embodiments, one or more computer-readable storage media embody encoded software.

Herein, reference to encoded software may encompass one or more applications, bytecode, one or more computer programs, one or more executables, one or more instructions, logic, machine code, one or more scripts, or source code, and vice versa, where appropriate, that have been stored or encoded in a computer-readable storage medium. In particular embodiments, encoded software includes one or more application programming interfaces (APIs) stored or encoded in a computer-readable storage medium. Particular embodiments may use any suitable encoded software written or otherwise expressed in any suitable programming language or combination of programming languages stored or encoded in any suitable type or number of computer-readable storage media. In particular embodiments, encoded software may be expressed as source code or object code. In particular embodiments, encoded software is expressed in a higher-level programming language, such as, for example, C, Perl, or a suitable extension thereof. In particular embodiments, encoded software is expressed in a lower-level programming language, such as assembly language (or machine code). In particular embodiments, encoded software is expressed in JAVA. In particular embodiments, encoded software is expressed in Hyper Text Markup Language (HTML), Extensible Markup Language (XML), or other suitable markup language. The foregoing description of embodiments of the disclosure has been presented for purposes of illustration and description. It is not intended to be exhaustive or to limit the disclosure to the precise form disclosed, and modifications and variations are possible in light of the above teachings or may be acquired from practice of the disclosure. The embodiments were chosen and described in order to explain the principals of the disclosure and its practical application to enable one skilled in the art to utilize the disclosure in various embodiments and with various modifications as are suited to the particular use contemplated. Other substitutions, modifications, changes and omissions may be made in the design, operating conditions and arrangement of the embodiments without departing from the scope of the present disclosure. Such modifications and combinations of the illustrative embodiments as well as other embodiments will be apparent to persons skilled in the art upon reference to the description. It is, therefore, intended that the appended claims encompass any such modifications or embodiments.

Depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. Although certain computer-implemented tasks are described as being performed by a particular entity, other embodiments are possible in which these tasks are performed by a different entity.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, the processes described herein can be embodied within a form that does not provide all of the features and benefits set forth herein, as some features can be used or practiced separately from others. The scope of protection is defined by the appended claims rather than by the foregoing description. All changes which come within the meaning and range of equivalency of the claims are to be embraced within their scope.

What is claimed is:

1. A method comprising:
receiving, by a user device, a code start trigger, wherein the user device is dockable on a crash cart and in communication with a controller disposed in relation to the crash cart;
responsive to the code start trigger:
initiating a code workflow;
creating an event log for the code workflow; and
determining a patient rhythm;
monitoring for real-time code events, the monitored real-time code events comprising patient intervention and a new patient rhythm; and
responsive to a determination that a real-time code event has occurred, executing programmed action comprising updating the event log.

2. The method of claim 1, comprising:
starting a timer for a timed intervention associated with the patient rhythm; and
visually indicating a real-time countdown of the timer on the user device.

3. The method of claim 2 comprising, responsive to a determination that the timed intervention has been completed:
restarting the timer for the timed intervention; and
visually indicating a real-time countdown of the restarted timer on the user device.

4. The method of claim 1, comprising:
starting a plurality of timers for a plurality of timed interventions associated the patient rhythm; and
visually indicating real-time countdowns of the plurality of timers on the user device.

5. The method of claim 1 comprising, responsive to the code start trigger, receiving an initial dataset for the code workflow, the receiving comprising:
on a user interface on the user device, pre-populating a code start time with a time associated with the code start trigger; and
allowing a user to modify the code start time.

6. The method of claim 1 comprising, providing a code user interface comprising a listing of patient interventions associated with the patient rhythm.

7. The method of claim 6 comprising, responsive to a determination of a new patient rhythm, updating the code user interface to include interventions associated with the new patient rhythm.

8. The method of claim 1 comprising, responsive to a determination of a code-stop event:
recording a code stop time corresponding to a time associated with the code-stop event; and
finalizing data related to the code workflow.

9. The method of claim 8, comprising the user device storing the finalized data on the controller.

10. The method of claim 8, the finalizing comprising providing an interface on the user device for a user to edit and correct the event log.

11. A crash cart comprising:
a plurality of drawers;
a removable user device docked to a surface of the crash cart, wherein the surface is above the plurality of drawers; and
an interior compartment under the surface of the crash cart and above the plurality of drawers, wherein the interior compartment is hidden from view when the removable user device is docked and is exposed when the removable user device is undocked.

12. The crash cart of claim 11, comprising:
a controller disposed in the crash cart and in communication with the removable user device; and
a sensor disposed in the crash cart and in communication with the controller, wherein the sensor is configured to measure at least one of temperature and humidity.

13. A non-transitory computer-program product comprising a computer-usable medium having computer-readable program code embodied therein, the computer-readable program code adapted to be executed to implement a method comprising:
receiving, by a user device, a code start trigger, wherein the user device is dockable on a crash cart and in communication with a controller disposed in relation to the crash cart;
responsive to the code start trigger:
initiating a code workflow;
creating an event log for the code workflow; and
determining a patient rhythm;
monitoring for real-time code events, the monitored real-time code events comprising patient intervention and a new patient rhythm; and
responsive to a determination that a real-time code event has occurred, executing programmed action comprising updating the event log.

14. The computer program product of claim 13, the method comprising:
starting a timer for a timed intervention associated with the patient rhythm; and
visually indicating a real-time countdown of the timer on the user device.

15. The computer program product of claim 14, the method comprising, responsive to a determination that the timed intervention has been completed:
restarting the timer for the timed intervention; and
visually indicating a real-time countdown of the restarted timer on the user device.

16. The computer program product of claim 13, the method comprising:
starting a plurality of timers for a plurality of timed interventions associated with the patient rhythm; and
visually indicating real-time countdowns of the plurality of timers on the user device.

17. The computer program product of claim 13, the method comprising, responsive to the code start rigger, receiving an initial dataset for the code workflow, the receiving comprising:
on a user interface on the user device, pre-populating a code start time with a time associated with the code start trigger; and
allowing a user to modify the code start time.

18. The computer program product of claim 13, the method comprising providing a code user interface comprising a listing of patient interventions associated with the patient rhythm.

19. The computer program product of claim 18, the method comprising, responsive to a determination of a new patient rhythm, updating the code user interface to include interventions associated with the new patient rhythm.

20. The computer program product of claim 13, the method comprising, responsive to a determination of a code-stop event:
recording a code stop time corresponding to a time associated with the code-stop event; and
finalizing data related to the code workflow.
